SEQUENCE LISTING (4 sequences)

SEQ ID NO: 1 - protein, 120 aa, Mus musculus
EVQLQESGPSLVKPSQTLSLTCSVTGDSITSDYWNWIRKFPGNKLESMGYITYSGTTYYNPSLKSRISITRDTSKNQYYLQLNSVTSEDTATYYCARGVDYEPSYYFDYWGQGTTLTVSS

SEQ ID NO: 2 - protein, 107 aa, mus musculus
DIQMTQTTSSLSASLGDRVTISCSASQGISNYLNWYQQKPDGTVKLLIYYTSSLHSGVPSRFSGSGSGTDYSLTISNLEPEDIATYYCQQYSNLPWTFGGGTKLEIK

SEQ ID NO: 3 - protein, 120 aa, mouse-human deimmunized chimera
EVQLQESGPSLVKPSQTLSLTCTVSGDSITSDYWNWIRQAPGKGLESMGYITYSGTTYYNPSLKSRVTISRDTSKNQYYMELSSLRSEDTATYYCARGVDYEPSYYFDYWGQGTLVTVSS

SEQ ID NO: 4 - protein, 107 aa, mouse-human deimmunized chimera
DIQMTQSPSSLSASVGDRVTITCSASQGISNYLNWYQQKPGKAPKLLIYYTSSLHSGVPSRFSGSGSGTDYSLTISSLQPEDIATYYCQQYSNLPWTFGQGTKVEIK

(19) United States
(12) Patent Application Publication
(10) Pub. No.: US 2009/0004183 A1
(43) Pub. Date: Jan. 1, 2009

(54) Compositions and Methods for Regulating the Alternative Pathway of Complement

(76) Inventors: Ronald P. Taylor, Keswick, VA (US); David John Dilillo, Durham, NC (US); Margaret A. Lindorfer, Keswick, VA (US); Andrew W. Pawluczkowycz, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 11/658,124

(22) PCT Filed: Jul. 25, 2005
(86) PCT No.: PCT/US05/26351

§ 371 Date: Jan. 22, 2007

Related U.S. Application Data
(60) Provisional application No. 60/658,540, filed on Mar. 4, 2005.
(60) Provisional application No. 60/590,514, filed on Jul. 23, 2004.

(51) Int. Cl. A61K 39/395; A61P 29/00; A61P 9/10; C07H 21/04; C12N 15/63; C12N 5/06; C07K 16/18

(52) U.S. Cl. 424/133.1; 424/141.1; 424/158.1; 536/23.53; 435/320.1; 530/389.3; 530/388.25

(57) ABSTRACT

The present invention provides compositions and methods for regulating the alternative complement pathway.

### CROSS REFERENCE TO RELATED APPLICATIONS

This application is entitled to priority pursuant to 35 U.S.C. § 119(e) to U.S. provisional patent application Nos. 60/590,514, filed Jul. 23, 2004, and 60/658,540, filed Mar. 4, 2005.

### BACKGROUND OF THE INVENTION

The complement system is part of the innate (non-adaptive) immune system and can produce an inflammatory and protective reaction in response to challenges from pathogens before an adaptive immune response can occur. Complement can also be activated in the body in the absence of an external threat, including conditions associated with ischemia-reperfusion injury, or as a consequence of certain autoimmune diseases (Boackle, S. A., et al., Current Directions in Autoimmunity, 2003, 6:154-168; Makrides, S. C., Pharm. Rev., 1998, 50:59-87; Riedemann, N. C., et al., Amer. J. Path., 2003, 162:363-367; Stahl, G. L., et al., Amer. J. Path., 2003, 162:449-455; Walport, M. J., N. Engl. J. Med., 2001, 344:1058-1066). In these latter cases, such activity can lead to substantial tissue damage, and therefore it would be therapeutically beneficial to modulate the activity of the complement system so such negative effects are diminished.

Discrete steps in the complement pathways can be targeted using monoclonal antibodies (mAbs), which can, in principle, either up-regulate or down-regulate these steps. For example, mAbs which bind to sites on complement proteins and promote stabilization of labile intermediates can enhance complement activation, conversely mAbs which induce dissociation or prevent formation of these intermediates can down-regulate complement (Mastellos, D., et al., Mol. Immunol., 2004, 40:1213-1221; Morgan, B. P., et al., Mol. Immunol., 2003, 40:159-170; Walport, M. J., N. Engl. J. Med., 2001, 344:1058-1066). Increasing evidence suggests that blocking activation of the alternative pathway (AP) of complement, while leaving the classical pathway (CP) and lectin pathway active, can prevent or reduce certain disease pathologies while maintaining host defense afforded by the other two pathways (Elliott, M. K., et al., Kidney Intl., 2004, 65:129-138; Holers, V. M., et al., Mol. Immunol., 2004, 41:147-152; Thurman, J. M., et al., Mol. Immunol., 2005, 42:87-97). Several mAbs specific for factor B have been reported, and recently Thurman et al. have demonstrated that an anti-factor B mAb that recognizes human, mouse, and several other species' factor B can indeed block the AP (Thurman, J. M., et al., Mol. Immunol., 2005, 42:87-97). This group has also demonstrated in a mouse model the effectiveness of the anti-factor B mAb in preventing fetal loss induced by IgG anti-phospholipid antibodies; here the loss is mediated by activation of the AP.

Mouse IgG1 mAb 3E7, specific for human C3b and iC3b, can enhance the immunotherapeutic action of Rituximab (RTX) when it binds to CD20-positive B cells in complement-replete human serum (Kennedy, A. D., et al., J. Immunol., 2004, 172:3280-3288; Kennedy, A. D., et al., Blood, 2003, 101:1071-1079). In particular, mAb 3E7 enhanced and prolonged deposition of C3b/iC3b on targeted cells, and increased RTX-promoted complement-mediated cell lysis.

Complement performs an important immunological role in the killing of pathogenic organisms and the generation of an optimal antibody response. Complement activation can be initiated by three different pathways: the classical pathway (CP), the alternative pathway (AP) and the lectin pathway. Each initiation pathway functions in common to cleave the serum protein C3 into two fragments. One fragment, C3a, is an anaphylactic agent, while the other fragment, C3b, binds covalently to activating targets, marking foreign substances for lysis and/or immune clearance.

Inappropriate activation of complement occurs in a large number of inflammatory, ischemic diseases, and recent findings have implicated the alternative pathway as playing a key role in such diseases. The role of the alternative pathway in generating activated pro-inflammatory fragments at extra-vascular sites is substantial.

One important and earliest step in the activation cascade that characterizes the alternative pathway of complement is the binding of activated C3b to Factor B, which is then followed by activation of factor B to Bb. Accordingly, one previously described approach to block the alternative pathway of complement is based on the use of mAbs directed toward Factor B as blocking agents, to prevent Factor B from binding to C3b.

There is a long felt need in the art for methods and compositions to regulate the alternative pathway of complement. The present invention satisfies these needs.

### SUMMARY OF THE INVENTION

The present invention provides compositions and methods for regulating the alternate complement pathway. In one aspect, the present invention provides compositions and methods for inhibiting activation of the alternate complement pathway. In another aspect, the present invention provides compositions and methods for inhibiting progression of the alternate complement pathway.

Because mouse IgG1 mAbs do not effectively activate human complement, studies described herein addressed the question of whether binding of mAb 3E7 to C3b/iC3b opsonized cells might interfere with binding of factors H or B, and thus influence processing of C3b-opsonized substrates by either the classical or alternative pathway. The experiments disclosed herein show that binding of mAb 3E7, and of a deimmunized chimeric, partially humanized form of the mAb, H17, to C3b-opsonized substrates inhibits binding of both factor H and factor B to these substrates. The consequence of this inhibition is that, although mAb 3E7 can allow and/or enhance the CP, it very effectively blocks activation of the AP of complement by inhibiting formation of the initial C3b convertase due to blocking the binding of factor B to C3(H20) or C3b.

Various aspects and embodiments of the invention are described in further detail below.

### BRIEF DESCRIPTION OF THE DRAWINGS

FIG. 1, comprising FIGS. 1A, 1B, 1C, and 1D, graphically depicts that the binding of A1488 factor H and factor B to C3b-zymosan or C3b-Sepharose is blocked by mAbs 3E7 and H17. FIG. 1A—Flow cytometric analysis of the binding of A1488 factor H (100 μg/ml) to C3b-zymosan in the presence and absence of mAb 3E7 or H17 at either 100 or 150 μg/ml. Values indicated are in molecules of equivalent soluble fluorochrome (MESF) units. FIG. 1B—C3b-opsonized zymosan was incubated with A1488 factor H (175 μg/ml) for 15 minutes, followed by addition of varying amounts of mAb 3E7 (1, 25 or 100 μg/ml). Then, 1, 10 and 30 minutes later, samples were washed and subjected to flow cytometry analysis. FIGS. 1C and 1D—A1488 factor B (50 μg/ml), factor D (2 μg/ml), Mg⁺² (5 mM), and properdin (20 μg/ml) were incubated for 30 min at 37° C. with C3b-opsonized zymosan (FIG. 1C) or Sepharose 4B (FIG. 1D) in the presence of 0, 10 and 100 μg/ml mAb 3E7 or mAb H17, and then binding was analyzed by flow cytometry (FIG. 1C) or by steady-state fluorescence (FIG. 1D). Naive substrate: not opsonized with C3b.

FIG. 2, comprising FIGS. 2A, 2B, and 2C, demonstrates graphically that AP-mediated C3b-opsonization of zymosan is blocked by mAbs 3E7 and H17. FIG. 2A—Zymosan was incubated, for either 15 minutes or 1 hour (60 minutes) at 37° C. with 10% NHS with and without mAb 3E7 (10 μg/ml). The samples were then washed and probed with two FITC-labeled C3b-specific mAbs, mAb 1H8 and mAb 7C12. C3b deposition was measured by flow cytometry; the background signals for no serum, mAb 1H8, and mAb 7C12 were less than 1700 MESF units. FIG. 2B—Zymosan was incubated with 25 and 50% NHS for 30 min at 37° C. with and without mAbs 3E7 (0, 50, 200 μg/ml) and H17 (50 and 200 μg/ml), and then probed as in A. FIG. 2C—Zymosan was mixed with 20 or 50% NHS in the presence and absence of mAbs 3E7 (0, 25, and 100 μg/ml) and H17 (0, 25, and 100 μg/ml). The mixture also contained 2% EA (“antibody-opsonized sheep erythrocytes”), which were not lysed as the buffer contained Mg-EGTA. After an incubation of 15 minutes at 37° C., samples were washed, the EA were lysed with distilled water, and after additional washes the samples were probed with A1488 mAb 1H8.

FIG. 3, comprising FIGS. 3A and 3B, demonstrates graphically that AP-mediated C3b-opsonization of Sepharose 4B is blocked by mAbs 3E7 and H17. FIG. 3A-Sepharose 4B was incubated with 75% NHS with or without varying amounts of mAbs 3E7 and H17 (37.5, 75, and 150 μg/ml). After incubation for 15 minutes at 37° C., reaction mixtures were washed, probed with FITC mAb 1H8 or mAb 7C12, washed and steady-state fluorescence was measured. FIG. 3B—Similar experiment as in 3A, but 50% NHS was used and samples were probed with FITC mAb 1H8.

FIG. 4, comprising FIGS. 4A, 4B, and 4C, demonstrates graphically that A1488 mAbs 3E7 and H117 show negligible binding to Sepharose 4B if they are present during the initial opsonization with NHS (FIG. 4A). FIGS. 4B and 4C demonstrate that added C3(H₂O) either produced in serum, or derived from the purified C3 molecule, binds to both mAbs 3E7 and H17, and thus can inhibit their abilities to block the alternative pathway. FIG. 4A—A1488 mAbs 3E7 and H17 show negligible binding to Sepharose 4B if they are present during the initial opsonization with NHS. Sepharose 4B was incubated with 50% NHS with and without A1488 mAb 3E7 or A1488 mAb H17 (100 μg/ml each). After incubation at 37° C. for 30 minutes, reaction mixtures were washed, and samples that did not contain mAbs during the opsonization were incubated with A1488 mAb 3E7 or A1488 mAb H17 for 30 minutes at 37° C., to verify C3b deposition. Steady-state fluorescence was measured. FIGS. 4B and 4C—NHS in which all C3 was converted to C3(H₂O) (FIG. 4B), or purified C3(H₂O) (FIG. 4C) blocks the inhibitory activity of mAbs 3E7 and H117 when the C3(H₂O) is incubated with the mAbs before they are tested in the alternative pathway activation assay. Varying amounts of KBr-treated NHS or KBr-treated purified C3 were incubated for 15 minutes with mAbs 3E7 or H17, and then combined with Sepharose 4B and 20% NHS. After incubation for 30 minutes at 37° C., samples were washed and probed with A1488 mAb 1H8. Different NHS pools were used in FIGS. 4B and 4C.

FIG. 5, comprising FIGS. 5A, 5B, and 5C, graphically illustrates dose responses of the ability of mAbs 3E7 and H117, but not 1H8 to prevent lysis of rabbit erythrocytes (E) in Mg-EGTA. FIG. 5A—NHS (final concentration 45%) was mixed with varying amounts of mAbs 3E7 and 1H8. After incubation for 1 hour at 37° C., mixtures were quenched with EDTA and the optical density of the supernatants was measured. FIGS. 5B and 5C— Dose-response experiments evaluating the ability of mAb 3E7 (5B) and mAb H17 (5C) to block AP-mediated lysis of rabbit E (0, 15, 30, 60, 120 μg/ml). Complete lysis corresponded to a final optical density of ˜1.6.

FIG. 6, comprising FIGS. 6A and 6B, graphically illustrates that mAb 3E7 effectively inhibits progression of the AP. FIG. 6A—Incubation mixtures of 50% NHS and zymosan were equilibrated at 37° C. and then quenched at varying times by combination with cold EDTA or with mAb 3E7 (see Materials and Methods). After 25 minutes, all reaction mixtures were treated with EDTA, washed, and probed with A1488 mAb 1H8. Groups include: 10 mM EDTA, 100 μg/ml mAb 3E7, 250 μg/ml 3E7, and buffer. FIG. 6B—The assay used in FIG. 6A was repeated. The background signal (mixtures quenched with 10 mM EDTA at time 0) in FIGS. 6A and 6B were 750 and 880 MESF, respectively.

FIG. 7 is a schematic representation of the DNA and amino acid sequence of the 3E7 murine monoclonal antibody heavy chain variable region. Restriction sites and coding regions are indicated.

FIG. 8 is a schematic representation of the DNA and amino acid sequence of the 3E7 murine monoclonal antibody light chain variable region. Restriction sites and coding regions are indicated.

FIG. 9, comprising FIGS. 9A and 9B, schematically represents the heavy chain variable region (9A) and light chain variable region (9B) amino acid sequences of H17. In FIG. 9A, the underlined regions indicate the amino acid residues which have been changed relative to 3E7. FIG. 9B represents the H17 light chain amino acid sequence and also compares the light chain region of H17 to 3E7, with the outlined letters in H17 indicating changes in amino acid residue relative to 3E7.

### DETAILED DESCRIPTION OF THE INVENTION

### Abbreviations and Acronyms

A1488—Alexa 488 fluorophore

AP—alternative pathway of complement

BSA—bovine serum albumin

CLL—chronic lymphocytic leukemia

CP—classical pathway of complement

E—erythrocyte

EA—antibody-opsonized sheep erythrocytes

eq—equation

FITC—fluorescein isothiocyanate

GVB—gelatin veronal-buffered saline

hr—hour(s)

mAbs—monoclonal antibodies

MESF—molecules of equivalent soluble fluorochrome

min—minute(s)

NHS—normal human serum

RTX—rituximab

### DEFINITIONS

In describing and claiming the invention, the following terminology will be used in accordance with the definitions set forth below.

As used herein, the articles “a” and “an” refer to one or to more than one, i.e., to at least one, of the grammatical object of the article. By way of example, “an element” means one element or more than one element.

What is meant by “activation of the alternative complement pathway” is the key first step in initiation of the alternative pathway of complement, which is the natural “turnover” of C3. Approximately 1% per hour of C3 reacts with water to form C3(H₂0). This molecule then binds Factor B, and then Factor D activates the binary complex, forming Bb·C3(H₂0). This is the initial activation step. This activate binary complex then can cut C3, forming C3b. The C3b will bind more Factor B, which in the presence of Factor D, forms Bb·C3b. This later binary complex allows “progression” and continuation of the alternative pathway, as it can cut more C3, forming even more C3b, etc. The reaction is now autocatalytic, in that the new C3b can combine with Factor B, etc. The key action of 3E7/H17 is that the mAbs bind to both C3(H₂0), and to C3b. In so doing, they prevent Factor B from binding, thus stopping both the activation and progression step. That is, neither binary complex can be formed.

A disease or disorder is “alleviated” if the severity of a symptom of the disease, condition, or disorder, or the frequency with which such a symptom is experienced by a subject, or both, are reduced.

What is meant by “alternative complement pathway mediated disease, disorder, or condition,” is a disease, disorder, or condition in which the alternative complement pathway plays a role, whether directly or indirectly. It does not mean that the alternative complement pathway causes the disease, disorder, or condition.

As used herein, an “analog” of a chemical compound is a compound that, by way of example, resembles another in structure but is not necessarily an isomer (e.g., 5-fluorouracil is an analog of thymine).

As used herein, “amino acids” are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

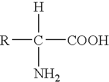

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as exchanges within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to a polypeptide, an isolated nucleic acid, or other agent used in the method of the invention.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell is a cell being examined.

A “pathoindicative” cell is a cell which, when present in a tissue, is an indication that the animal in which the tissue is located (or from which the tissue was obtained) is afflicted with a disease or disorder.

A “pathogenic” cell is a cell which, when present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A disease, condition, or disorder is “alleviated” if the severity of a symptom of the disease or disorder, the frequency with which such a symptom is experienced by a patient, or both, are reduced.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “inhibit,” as used herein, refers to the ability of a compound of the invention to reduce or impede a described function. Preferably, inhibition is by at least 10%, more preferably by at least 25%, even more preferably by at least 50%, and most preferably, the function is inhibited by at least 75%.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

The term “peptide” typically refers to short polypeptides.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

A “recombinant polypeptide” is one which is produced upon expression of a recombinant polynucleotide.

A peptide encompasses a sequence of 2 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR— linkages (bonds) have been replaced by a non-peptidyl linkage such as a —CH2-carbamate linkage (—CH2OC(O)NR—), a phosphonate linkage, a —CH2-sulfonamide (—CH2-S(O)2NR—) linkage, a urea (—NHC(O)NH—) linkage, a—CH2-secondary amine linkage, or with an alkylated peptidyl linkage (—C(O)NR—) wherein R is C1-C4 alkyl;

2. peptides wherein the N-terminus is derivatized to a—NRR1 group, to a —NRC(O)R group, to a —NRC(O)OR group, to a —NRS(O)₂R group, to a —NHC(O)NHR group where R and R1 are hydrogen or C1-C4 alkyl with the proviso that R and R1 are not both hydrogen;

3. peptides wherein the C terminus is derivatized to —C(O)R2 where R2 is selected from the group consisting of C1-C4 alkoxy, and —NR3R4 where R3 and R4 are independently selected from the group consisting of hydrogen and C1-C4 alkyl.

The term “permeability,” as used herein, refers to transit of fluid, cell, or debris between or through cells and tissues.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

By the term “specifically binds,” as used herein, is meant an antibody which recognizes and binds a specific protein, but does not substantially recognize or bind other molecules in a sample, or it means binding between two or more proteins as in part of a cellular regulatory process, where said proteins do not substantially recognize or bind other proteins in a sample.

The term “standard,” as used herein, refers to something used for comparison. For example, it can be a known standard agent or compound which is administered or added to a control sample and used for comparing results when measuring said compound in a test sample. Standard can also refer to an “internal standard,” such as an agent or compound which is added at known amounts to a sample and is useful in determining such things as purification or recovery rates when a sample is processed or subjected to purification or extraction procedures before a marker of interest is measured.

A “subject” of diagnosis or treatment is an animal, including a human.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

As used herein, the term “treating” includes prophylaxis of the specific disease, disorder, or condition, or alleviation of the symptoms associated with a specific disease, disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

### EMBODIMENTS OF THE INVENTION

One embodiment of the present invention provides compositions and methods for regulating the alternative pathway of complement. In one aspect, the present invention provides compositions and methods for inhibiting activation of the alternative pathway of complement. In another aspect, the present invention provides compositions and methods for inhibiting progression of the alternative pathway of complement. In one aspect, the invention provides antibodies directed against C3, C3(H₂O), and C3b, or homologs, fragments, or derivatives thereof, which inhibit the alternative complement pathway. In another aspect, the antibodies are monoclonal antibodies, or homologs, derivatives, chimeras, or fragments thereof. In yet another aspect of the invention, the monoclonal antibodies, or homologs, derivatives, or fragments thereof, are mAbs 3E7 and H17. In one aspect monoclonal antibodies of the present invention have activities similar to those of 3E7 and H17. One of ordinary skill in the art would appreciate that, depending on the species of animal in which the alternative complement pathway is to inhibited, antibodies with appropriate specificities for C3, C3(H₂O), and C3b will need to be prepared.

In one embodiment, the antibodies of the invention comprise amino acid sequences selected from the group consisting of SEQ ID NOs:1, 2, 3, and 4 (see the Examples section entitled “Antibody Sequences”). In one aspect, an amino acid sequence of the variable region of an antibody of the invention shares at least 50% sequence identity with a sequence selected from the group consisting of SEQ ID NOs:1, 2, 3, and 4. In another aspect, an amino acid sequence of the variable region of an antibody of the invention shares at least 75% sequence identity with a sequence selected from the group consisting of SEQ ID NOs:1, 2, 3, and 4. In a further aspect, an amino acid sequence of the variable region of an antibody of the invention shares at least 85% sequence identity with a sequence selected from the group consisting of SEQ ID NOs:1, 2, 3, and 4. In yet another aspect, an amino acid sequence of the variable region of an antibody of the invention shares at least 90% identity with a sequence selected from the group consisting of SEQ ID NOs:1, 2, 3, and 4. In another aspect, an amino acid sequence of the variable region of an antibody of the invention shares at least 95% identity with a sequence selected from the group consisting of SEQ ID NOs:1, 2, 3, and 4.

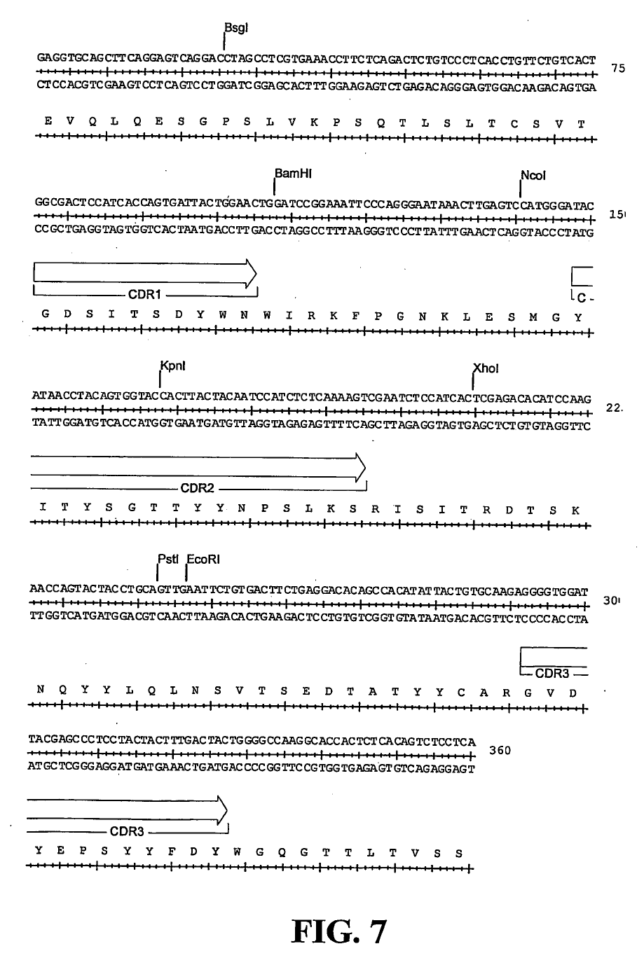

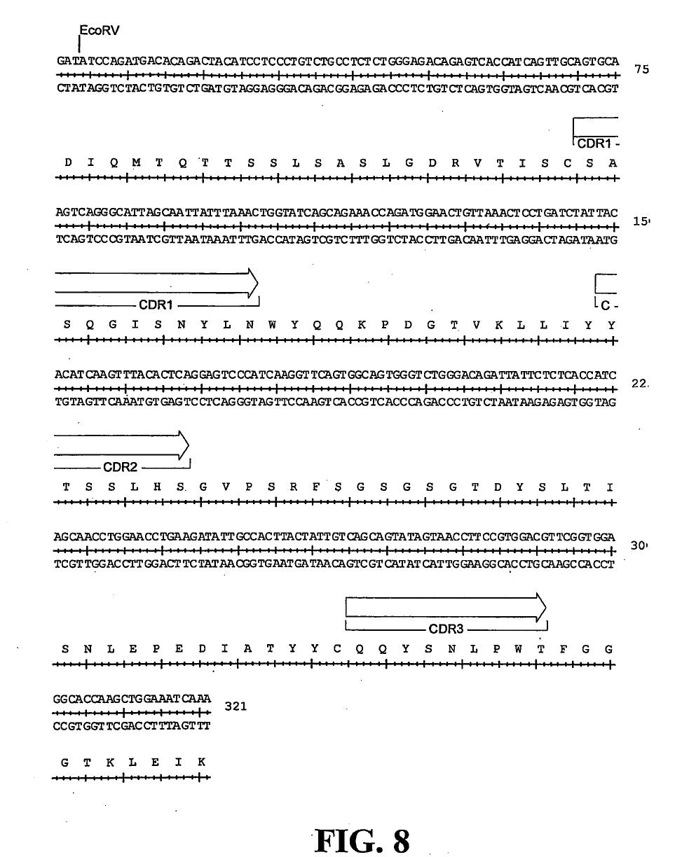

In one embodiment, the present invention provides isolated nucleic acids comprising nucleic acid sequences encoding the antibodies of the invention, or homologs, derivatives, chimeras, or fragments thereof. In one aspect, the isolated nucleic acids comprise the nucleic acid sequences of FIGS. 7 and 8. In one aspect, the nucleic acid sequences comprise sequences encoding peptides comprising SEQ ID NOs: selected from the group consisting of SEQ ID NOs:1, 2, 3, and 4. In one aspect, the invention provides a host cell comprising a nucleic acid sequence encoding a peptide comprising an amino acid sequence selected from the group consisting of SEQ ID NOs:1, 2, 3, and 4.

In one embodiment, the invention provides compositions and methods for inhibiting the alternative pathway of complement by blocking factor D/properdin-mediated binding of factor B to C3b-opsonized zymosan and sepharose.

In one embodiment, the invention provides compositions and methods for inhibiting the alternative pathway of complement by blocking factor H to C3b.

In one embodiment, the invention provides methods for diagnosing and treating diseases, conditions, and disorders associated with, or affected by, the alternative complement pathway, in a subject. In one aspect, the diseases, conditions, and disorders, include, but are not limited to, inflammatory diseases, conditions, and disorders and ischemic diseases, disorders, and conditions. In one aspect, the compounds of the present invention inhibit cell lysis induced by the alternative pathway of complement. In one embodiment, the invention provides compositions and methods for treating tissue injury. In one aspect, the tissue injury is acute. Such injuries include, but are not limited to, ischemia reperfusion injury associated with kidney injury, cardiac injury such as myocardial infarction, transplantation, and cardiopulmonary bypass.

In one embodiment, the subject is an animal. In one aspect, the animal is a human.

The present invention also provides methods for identifying regulators of the alternative complement pathway.

Antibodies directed against proteins, polypeptides, or peptide fragments thereof of the invention may be generated using methods that are well known in the art. For instance, U.S. patent application Ser. No. 07/481,491, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For the preparation of monoclonal antibodies, any technique which provides for the production of antibody molecules by continuous cell lines in culture may be utilized. For example, the hybridoma technique originally developed by Kohler and Milstein (1975, Nature 256:495-497), the trioma technique, the human B-cell hybridoma technique (Kozbor et al., 1983, Immunology Today 4:72), and the EBV-hybridoma technique (Cole et al., 1985, in Monoclonal Antibodies and Cancer Therapy, Alan R. Liss, Inc., pp. 77-96) may be employed to produce human monoclonal antibodies. In another embodiment, monoclonal antibodies are produced in germ-free animals utilizing the technology described in international application no. PCT/US90/02545, which is incorporated by reference herein in its entirety.

In accordance with the invention, human antibodies may be used and obtained by utilizing human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, Inc., pp. 77-96). Furthermore, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for desired epitopes together with genes from a human antibody molecule of appropriate biological activity can be employed; such antibodies are within the scope of the present invention. Once specific monoclonal antibodies have been developed, the preparation of mutants and variants thereof by conventional techniques is also available.

In one embodiment, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the invention.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein may be cloned and sequenced using technology which is available in the art, and is described, for example, in Wright et al. (1992, Critical Rev. in Immunol. 12(3,4): 125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

In the production of antibodies, screening for the desired antibody can be accomplished by techniques known in the art, e.g., ELISA (enzyme-linked immunosorbent assay). Antibodies generated in accordance with the present invention may include, but are not limited to, polyclonal, monoclonal, chimeric (i.e., “humanized”), and single chain (recombinant) antibodies, Fab fragments, and fragments produced by a Fab expression library.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters. Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide. Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation,” a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

The present invention also provides for homologs of proteins and peptides. Homologs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on peptide function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Homologs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The present invention also provides nucleic acids encoding peptides, proteins, and antibodies of the invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

It is not intended that the present invention be limited by the nature of the nucleic acid employed. The target nucleic acid may be native or synthesized nucleic acid. The nucleic acid may be from a viral, bacterial, animal or plant source. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89 (polylysine condensation of DNA in the form of adenovirus).

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

In some circumstances, as where increased nuclease stability is desired, nucleic acids having modified internucleoside linkages may be preferred. Nucleic acids containing modified internucleoside linkages may also be synthesized using reagents and methods that are well known in the art. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH2-S—CH2), dimethylene-sulfoxide (—CH2-SO—CH2), dimethylene-sulfone (—CH2-SO2-CH2), 2′-O-alkyl, and 2′-deoxy-2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein).

Oligonucleotides which contain at least one phosphorothioate modification are known to confer upon the oligonucleotide enhanced resistance to nucleases. Specific examples of modified oligonucleotides include those which contain phosphorothioate, phosphotriester, methyl phosphonate, short chain alkyl or cycloalkyl intersugar linkages, or short chain heteroatomic or heterocyclic intersugar (“backbone”) linkages. In addition, oligonucleotides having morpholino backbone structures (U.S. Pat. No. 5,034,506) or polyamide backbone structures (Nielsen et al., 1991, Science 254: 1497) may also be used.

The examples of oligonucleotide modifications described herein are not exhaustive and it is understood that the invention includes additional modifications of the antisense oligonucleotides of the invention which modifications serve to enhance the therapeutic properties of the antisense oligonucleotide without appreciable alteration of the basic sequence of the antisense oligonucleotide.

The nucleic acids may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size of the DNA to be purified.

The term nucleic acid also specifically includes nucleic acids composed of bases other than the five biologically occurring bases (adenine, guanine, thymine, cytosine and uracil).

The present invention is also directed to pharmaceutical compositions comprising the vascular permeability regulatory compounds of the present invention. More particularly, such compounds can be formulated as pharmaceutical compositions using standard pharmaceutically acceptable carriers, fillers, solublizing agents and stabilizers known to those skilled in the art.

The invention is also directed to methods of administering the compounds of the invention to a subject.

Pharmaceutical compositions comprising the present compounds are administered to an individual in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

The invention also encompasses the use pharmaceutical compositions of an appropriate compound, homolog, fragment, analog, or derivative thereof to practice the methods of the invention, the composition comprising at least one appropriate compound, homolog, fragment, analog, or derivative thereof and a pharmaceutically-acceptable carrier.

The pharmaceutical compositions useful for practicing the invention may be administered to deliver a dose of between 1 ng/kg/day and 100 mg/kg/day. Pharmaceutical compositions that are useful in the methods of the invention may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the invention.

Compounds which are identified using any of the methods described herein may be formulated and administered to a subject for treatment of the diseases disclosed herein.

The invention encompasses the preparation and use of pharmaceutical compositions comprising a compound useful for treatment of the conditions, disorders, and diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology. A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may also be prepared, packaged, or sold in a formulation suitable for rectal administration, vaginal administration, parenteral administration

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self propelling solvent/powder dispensing container such as a device comprising the active ingredient dissolved or suspended in a low-boiling propellant in a sealed container.

Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein. A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference. Typically, dosages of the compound of the invention which may be administered to an animal, preferably a human, range in amount from 1 μg to about 100 g per kilogram of body weight of the subject. While the precise dosage administered will vary depending upon any number of factors, including but not limited to, the type of animal and type of disease state being treated, the age of the animal and the route of administration. Preferably, the dosage of the compound will vary from about 10 μg to about 10 g per kilogram of body weight of the animal. More preferably, the dosage will vary from about 10 mg to about 1 g per kilogram of body weight of the subject.

The compound may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the subject, etc.

The invention also includes a kit comprising a compound of the invention and an instructional material which describes administering the composition to a cell or a tissue of a subject. In another embodiment, this kit comprises a (preferably sterile) solvent suitable for dissolving or suspending the composition of the invention prior to administering the compound to the subject. The invention also provides an applicator, and an instructional material for the use thereof.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

The invention is now described with reference to the following examples. These examples are provided for the purpose of illustration only and the invention should in no way be construed as being limited to these examples, but rather should be construed to encompass any and all variations which become evident as a result of the teachings provided herein.

### General Methods:

### Antibodies and Complement Reagents

Anti-C3b mAbs 1H8, 3E7, and 7C12 have been described (Kennedy, A. D., et al., J. Immunol., 2004, 172:3280-3288; Kennedy, A. D., et al., Blood, 2003, 101:1071-1079). The three mAbs bind to different epitopes and do not cross-compete in binding assays. The hybridoma comprising the 3E7 monoclonal antibody has been deposited with the American Type Culture Collection (“ATCC”) depository and has patent deposit designation number PTA-090

A chimeric form of mAb 3E7, useful for clinical applications, was constructed by replacing the mouse Fc region with the human IgG1 Fc region using standard methods (Orlandi, R., et al., Proc. Natl. Acad. Sci., 1989, 86:3833-3837). The mAb, designated as H17, was further modified by de-immunization using technology developed by Biovation (UK) (Peng, W., et al., Canc. Immunol. Immunopath., 2005, in press) in which amino acid substitutions (16 in the heavy chain, and 12 in the light chain) were made to avoid recognition by T cells. Vectors containing the genes of variable domains, V_{H} and V_{L}, of mouse Mab 3E7, after verification of the DNA sequences, were transferred into eukaryotic expression vectors containing human γ1 and K constant regions, respectively. Those vectors were co-transfected into NS0 cells by electroporation and clones were screened for production of human IgG in culture supernatants via a human IgG1/κ ELISA. The highest producing clone was selected as the cell line to produce chimeric 3E7. For a more complete description of H17, see Peng et al., Cancer Immunology, Immunotherapy, 2005 (published online, Apr. 22, 2005).

Utilizing its proprietary modeling technique peptide threading, Biovation (UK) determined which mouse variable region sequences had the potential to bind to human MHC class II and elicit an immune response. Some or all of such sequences in the mouse variable region were then mutated by overlapping PCR into non-immunogenic sequences (DeImmunization). The modified variable regions were then spliced to the human γ1 constant regions and the constructs were transfected into NS0 cells for expression of DeImmunized chimeric 3E7s.

Rituximab (RTX) was purchased from the University of Virginia Hospital pharmacy. FITC-labeled mAbs were prepared following the manufacturer's instructions (Sigma, St. Louis, Mo.). Zymosan A (Sigma) was prepared by dispersion at 8 mg/ml in phosphate buffered-saline (PBS) and immersion in a boiling water bath for 10 minutes.

Sheep and rabbit erythrocytes (E) were obtained from Lampire Biological Laboratories (Pipersville, Pa.). Sheep hemolysin was obtained from Sigma. Purified C3, factors H, I, and D, and properdin were purchased from Advanced Research Technologies (La Jolla, Calif.). Factors H and B, and mAbs 7C12, 1H8, 3E7, and H17 were labeled with Alexa 488 (A1488) fluorophore following the manufacturer's instructions (Molecular Probes, Eugene, Oreg.). Sepharose 4B (Amersham Pharmacia Biotech, Uppsala, Sweden) was washed three times with borate-saline (BS) buffer and resuspended in BS buffer to give a 33% dispersion. Normal human serum (NHS) and chronic lymphocytic leukemia (CLL) patient blood was obtained with written informed consent. Most experiments reported with NHS are based on findings with three different pools prepared from sera taken from four or more individuals.

### B Cell Opsonization

Blood samples containing malignant B cells were obtained from CLL patients before and after treatment with RTX. Whole blood from the pre-treatment samples was anti-coagulated with EDTA, washed and then opsonized with ABO blood type-matched NHS containing 10 μg/ml RTX in the presence and absence of 10 μg/ml mAb 3E7. Alternatively, the washed blood was opsonized with autologous serum (post-RTX infusion, containing >100 μg/ml RTX), along with matched NHS as a complement source, +mAb 3E7. The matched NHS was needed because infusion of RTX led to substantial complement consumption in the patient (Kennedy, A. D., et al., J. Immunol., 2004, 172:3280-3288; Kennedy, A. D., et al., Blood, 2003, 101:1071-1079). The cells were incubated for 30 min at 37° C., and after three washes probed with A1488 mAbs specific for either C3b/iC3b (mAb 7C12) or for C3b/iC3b/C3dg (mAb 1H8) as well as with appropriate B cell markers (Kennedy, A. D., et al., J. Immunol., 2004, 172:3280-3288; Kennedy, A. D., et al., Blood, 2003, 101:1071-107. The E were then lysed and the samples were fixed and analyzed by flow cytometry. Flow cytometry was accomplished on a FACSCalibur flow cytometer (Becton-Dickinson, San Jose, Calif.), and in all measurements mean fluorescence intensities were converted to molecules of equivalent soluble fluorochrome (MESF) using calibrated standard beads (Spherotech, Libertyville, Ill.) (Kennedy, A. D., et al., J. Immunol., 2004, 172:3280-3288; Kennedy, A. D., et al., Blood, 2003, 101:1071-107). A similar procedure was used to opsonize Raji cells, except only fresh NHS and RTX, +mAb 3E7, were used in the opsonization step, which was conducted for either 1 or 24 hours.

### Alternative Pathway Assays

Unless otherwise specified, assays of the AP were conducted in mixtures or solutions containing NHS and substrates a-c (below) in Mg-EGTA buffer (gelatin veronal-buffered saline (GVB) (Pangburn et al., 1981) plus Mg²⁺ and EGTA). The final concentrations of Mg⁺ and EGTA were 5 mM and 8 mM, respectively.

a. Zymosan: Zymosan, NHS-Mg-EGTA, and mAb inhibitors were constituted to volumes of 100 μl; the final concentration of zymosan A was held at 0.8 mg/ml, and the final NHS concentration varied between 0 and 75%. Samples were incubated at 37° C. for periods between 15 minutes and 1 hour, quenched with the addition of EDTA (final concentration 10 mM), washed three times with PBS containing 1% bovine serum albumin (BSA-PBS) and probed with A1488 mAbs specific for C3 fragments for 30 minutes at either room temperature or at 37° C. (results were identical at both temperatures). The particles were then washed twice, fixed with 1% paraformaldehyde and analyzed by flow cytometry.

b. Sepharose 4B. A similar protocol was followed, except that 20 μl of 33% Sepharose 4B replaced zymosan A in the incubation mixture with NHS and inhibitors. After incubation at 37° C., the samples were treated with EDTA (10 mM final concentration), washed three times with BSA-PBS, and probed for 30 min at 37° C. with the A1488 mAbs. Samples were washed three times and the fluorescence signal measured in a VICTOR2 fluorimeter (Wallac, Turku, Finland), giving an average steady-state fluorescence reading for the Sepharose dispersion.

c. Rabbit E. Whole rabbit blood, collected in Alsevers, was washed three times with cold GVB, the buffy coat removed, and the E resuspended to a final hematocrit of 2% in Mg-EGTA buffer. Duplicate or triplicate 25 μl aliquots of the rabbit E, varying amounts of NHS, and the mAb inhibitors were mixed to give a final volume of 100 μl. Samples were incubated in a V-bottom, 96-well plate for 1 hour at 37° C., then 100 μl of cold GVB-20 mM EDTA was added to stop lysis. The plate was centrifuged, the supernatants isolated and the degree of E lysis determined by measuring the optical density at 405 nm. Controls for background lysis included samples with no serum. Complete lysis was achieved in the absence of inhibitors at 45% NHS, and results are expressed as degree of lysis relative to this value.

### Effects of C3(H2O)/(Inactive C3) on the Activity of mAb 3E7

One volume of either purified C3 (1 mg/ml) or NHS (presumed to be 1 mg/ml in C3) was mixed with an equal volume of 4 M KBr and incubated at 37° C. for one hour, and held overnight at 4° C. The mixture was then dialyzed against BS and used as a source of C3(H2O), an inactive C3 molecular species in which the internal thioester bond of C3 is inactivated by reaction with water (Muller-Eberhard, H. J., Ann. Rev. Bioch., 1988, 57:321-347; Oran, A., et al., J. Biol. Chem., 1999, 274:5120-5130; Pangburn, M. K., et al., J. Exp. Med., 1981, 154:856-867). Aliquots of the KBr-treated C3 or serum were then incubated with mAb 3E7 to test the ability of the C3(H2O) to bind to mAb 3E7 and thus block its potential to inhibit the AP, as manifested by restored AP-mediated generation of C3b-Sepharose.

### Classical Pathway Assays

Sheep E were opsonized with hemolysin (to produce EA) by combining 2 ml 50% sheep E, in GVB-10 mM EDTA, with 1 ml hemolysin solution; the mixture was incubated for 15 minutes at 37° C. (Whaley, K. et al., Eds.: Dodds, A. W. and Sim, R. B., Complement: A Practical Approach, 1997, IRL at Oxford University Press, 1948). Following incubation, the opsonized E (EA) were washed 3 times with GVB. NHS-mediated lysis of EA was examined in the presence and absence of the inhibitory mAbs using the above procedures except with [Mg²⁺]=0.5 mM and [Ca²⁺]=0.15 mM, and no EGTA.

Inhibition of Binding of A1488 Factor H or Factor B to C3b-Opsonized Substrates

C3b-opsonized zymosan (see above) was incubated with A1488 factor H (100 μg/ml), in the presence and absence of mAbs 3E7 or H17 in BSA-PBS for 30 minutes at 37° C., and then after three washes, binding was determined by flow cytometry. Alternatively, A1488 factor H (100 μg/ml) was first bound to C3b-opsonized zymosan for 15 minutes at room temperature; the sample was not washed, and then varying amounts of mAb 3E7 (or buffer) were added, and the mixtures were held at room temperature for varying time periods before washing and analysis by flow cytometry. In order to measure binding of A1488 factor B, C3b-opsonized Sepharose 4B or C3b-opsonized zymosan was mixed with A1488 factor B (50 μg/ml, in the presence and absence of mAbs 3E7 or H17) along with 5 mM Mg⁺², factor D and properdin, at 2 μg/ml and 20 μg/ml, respectively. After an incubation of 30 minutes at 37° C., the samples were washed, and then binding to C3b-zymosan and C3b-Sepharose was determined by flow cytometry or by steady state fluorescence.

### Kinetics of C3b Deposition on ZYmosan in the Presence of mAb 3E7

Reaction mixtures containing 50% NHS-Mg-EGTA and zymosan (0.8 mg/ml) were prepared and held at 37° C. in a water bath. Aliquots were removed at various times and combined with either buffer or mAb 3E7 to give final concentrations of 100 μg/ml or 250 μg/ml mAb, and these samples were held at 37° C. Alternatively, aliquots were removed and immediately quenched by addition of EDTA (final concentration 10 mM) and held on ice. After 25 minutes, reaction mixtures from the 37° C. incubations were quenched with EDTA and placed on ice. All samples were then washed three times with BSA-PBS and probed with A1488 mAb 1H8 for 30 minutes at 37° C. and then analyzed by flow cytometry to measure C3b deposition.

### Results

### Initial Findings: mAb 3E7 Inhibits Generation of C3dg on RTX-Opsonized B Cells

First examined was the ability of mAb 3E7 to stabilize C3b/iC3b generated on RTX-opsonized B cells by the CP of complement (Kennedy, A. D., et al., Blood, 2003, 101:1071-1079). Naive malignant B cells were opsonized in NHS containing RTX in the presence and absence of 10 μg/ml mAb 3E7. In an alternative test of the paradigm, isolated post-treatment patient serum containing RTX which was supplemented with NHS as a source of complement was used (Kennedy, A. D., et al., J. Immunol., 2004, 172:3280-3288). The malignant B cells were obtained from a CLL patient treated with RTX, taken just before (naïve B cells) and soon after (serum source) RTX infusion. The samples were reacted for 30 minutes at 37° C., and after washing probed with either A1488 mAb 7C12, which recognizes C3b/iC3b, or with A1488 mAb 1H8, which binds to C3b/iC3b/C3dg. Alternatively, Raji cells were opsonized with 50% NHS and RTX in the presence and absence of mAb 3E7, and after 1 and 24 hours, the cells were similarly analyzed.

The results in Table 1 indicate that the readout with mAb 1H8 (which recognizes all three forms of cell-bound C3b fragments) was approximately constant in the presence and absence of mAb 3E7. However, binding of mAb 7C12 was considerably enhanced and usually increased two-fold when mAb 3E7 was present during the opsonization step with RTX and a complement source (Table 1) (Kennedy, A. D., et al., Blood, 2003, 101:1071-1079). Without wishing to be bound by any particular theory, these findings, which were obtained using either RTX+matched NHS or patient serum containing RTX supplemented with matched NHS, suggest that mAb 3E7 inhibited degradation of C3b/iC3b to C3dg, perhaps by binding to C3b/iC3b at the site recognized by factor H, thus inhibiting the proteolytic cleavage mediated by factor I. The fact that relatively small amounts of mAb 3E7 were effective in the assays indicates it must bind weakly to native C3 which would otherwise block its action (Kennedy, A. D., et al., Blood, 2003, 101:1071-1079).

TABLE 1
mAb 3E7 Stabilizes C3b/iC3b on CLL and
Raji Cells Opsonized with RTX in Serum^{a}

| | | mAb Probe-C3 Species Deposition (MESF) | | | |
|---|---|---|---|---|---|
| | | mAb 7C12 | | mAb 1H8 | |
| | | C3b/iC3b | | C3b/iC3b/C3dg | |
| | Opsonization^{a} | | | | |
| | | | 25% | | 25% |
| | | 50% NHS, | autol- | 50% NHS, | autol- |
| | mAb | 10 μg/ml | ogous, | 10 μg/ml | ogous, |
| Cells | 3E7 | RTX | 25% NHS | RTX | 25% NHS |
| CLL patient 9 | − | 49,000 | 33,000 | 287,000 | 241,000 |
| Week 1 | | | | | |
| | + | 94,000 | 61,000 | 281,000 | 197,000 |
| CLL patient 9 | − | 28,000 | 26,000 | 97,000 | 133,000^{b} |
| Week 3 | | | | | |
| | + | 61,000 | 65,000 | 134,000 | 138,000 |
| CLL patient 9 | − | | 20,000 | | 100,000^{b} |
| Week 4 | | | | | |
| | + | | 55,000 | | 103,000 |
| Raji cells | − | 1,300,000 | | 3,000,000 | |
| 1 hr | | | | | |
| | + | 1,900,000 | | 3,100,000 | |
| Raji cells | − | 650,000 | | 3,200,000 | |
| 24 hr | | | | | |
| | + | 1,600,000 | | 3,500,000 | |

^{a}Opsonizations were either in 50% NHS + 10 μg/ml RTX, or in 25% patient autologous serum (containing >100 μg/ml RTX) plus 25% NHS as a complement source.
^{b}The decrease in C3 fragment deposition from week 1 to weeks 3 and 4 was likely due to progressive loss of CD20 from B cells induced by the high RTX doses (Kennedy, A. D., et al., J. Immunol., 2004, 172: 3280-3288; Kennedy, A. D., et al., Blood, 2003, 101: 1071-1079), thus leading to less RTX binding, and less complement activation.

mAbs 3E7 and H17 Compete with both Factor H and Factor B in Binding to C3b-opsonized Substrates

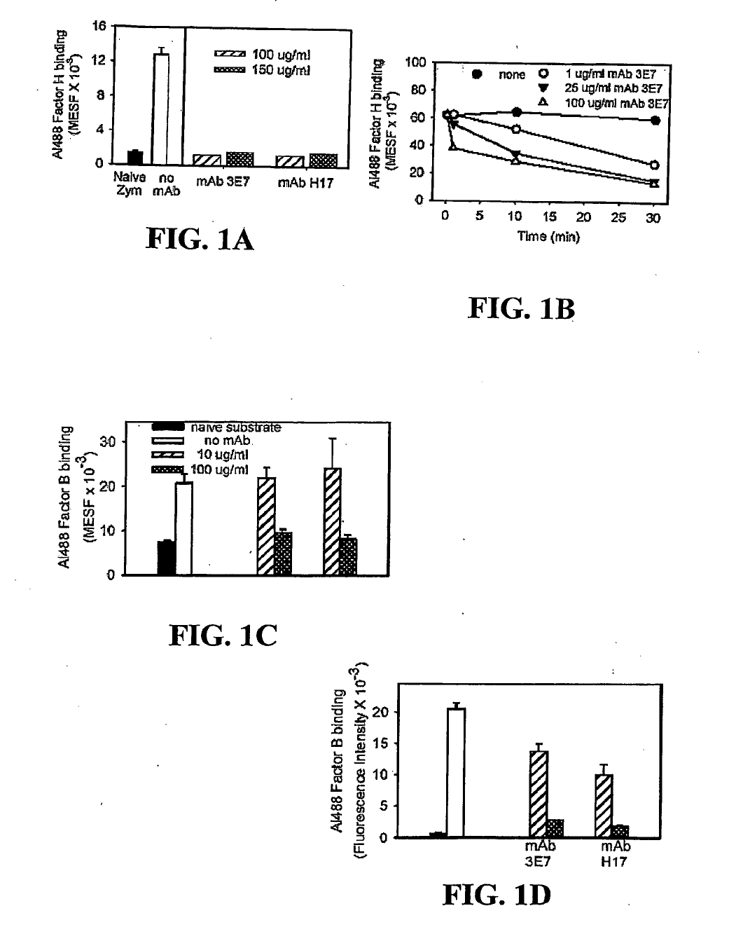

To test the hypothesis that mAb 3E7 inhibits the degradation of C3b/iC3b by competing with factor H for the same or a closely aligned site, we used flow cytometry to examine the ability of mAb 3E7 to inhibit binding of A1488 factor H to C3b-opsonized zymosan. Both mAb 3E7 and its chimeric, de-immunized form H17 are able to inhibit binding of factor H to C3b fragments (FIG. 1A). Moreover, kinetic studies demonstrate that relatively low concentrations of mAb 3E7 can induce dissociation of pre-bound factor H from C3b-opsonized zymosan (FIG. 1B).

Equations 1 and 2 respectively summarize the key steps in the initiation (equation 1) and amplification (equation 2) of the AP of complement. The disclosed experiments were designed to test the hypotheses—“by binding to C3(H2O) and to C3b, mAbs 3E7 and H17 block binding of factor B and/or inhibit conversion of factor B to Bb.” Such inhibition would block all downstream steps in the AP, including deposition of C3b on and/or lysis of substrates.

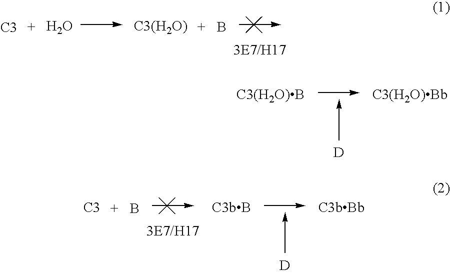

Several lines of evidence indicate that factor H and factor B bind to the same site on C3b (Becherer, J. D., et al., Biochemistry, 1992, 31:1787-1794; Farries, T. C., et al., Inflamm, 1990, 7:30-41; Koistinen, V., et al., Complement Inflamm., 1989, 6:270-280; Lambris, J. D., et al., J. Immunol., 1996, 156:4821-4832), and therefore the ability of mAb 3E7 to block binding of factor B to two different C3b-opsonized substrates, zymosan and Sepharose 4B, was tested. A1488 factor B alone bound poorly to either C3b-opsonized substrate (not shown), and required the presence of Mg+2, factor D and properdin. Presumably, under these conditions weakly bound factor B is converted to Bb in the presence of factor D and properdin (Muller-Eberhard, H. J., Ann. Rev. Bioch., 1988, 57:321-347; Thurman, J. M., et al., Mol. Immunol., 2005, 42:87-97); the resultant Bb molecule then binds to C3b with a higher affinity. We found that mAb 3E7 and H17 substantially blocked factor D/properdin-mediated binding of factor B to C3b-opsonized zymosan (FIG. 1C) and Sepharose 4B (FIG. 1D) (equation 2).

mAbs 3E7 and H17 Block the Alternative Pathway.

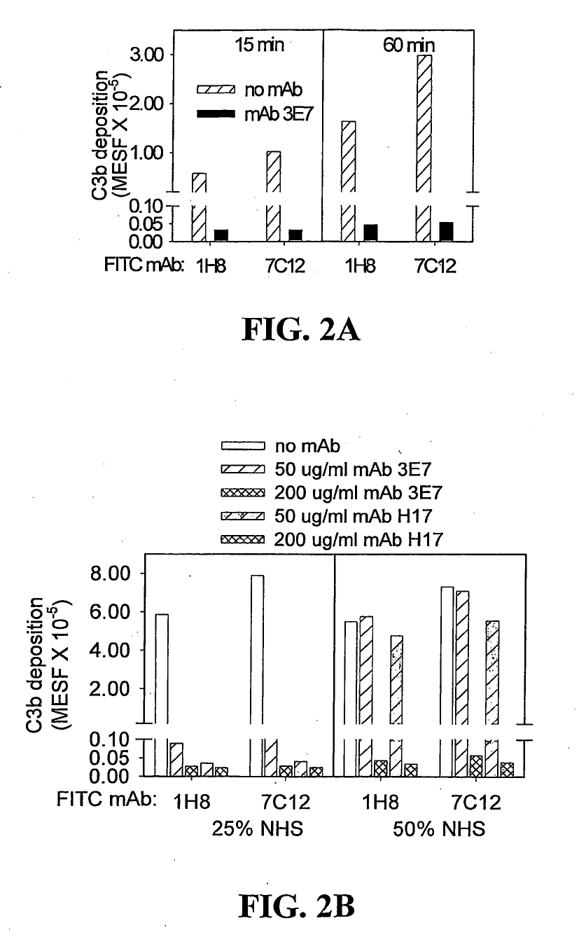

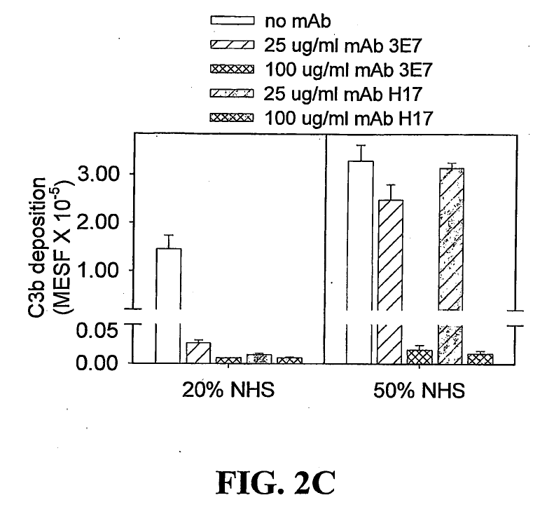

Based on the above observations, the potential of mAb 3E7 to inhibit serum-mediated deposition of C3b on zymosan, a reaction promoted by the AP (Pangburn, M. K., et al., J. Immunol., 1983, 131:1930-1935) was tested. Anti-C3b mAbs A1488 mAb 1H8 and A1488 mAb 7C12 were used to evaluate the amount of C3b deposition (as in Table 1). The results demonstrate that over a variety of conditions (10-50% NHS, 15 minutes to 1 hour) mAbs 3E7 and H17 inhibit AP-mediated deposition of C3b fragments on zymosan (FIGS. 2A and 2B). Dose-response experiments indicated that more inhibitory mAb is needed to block C3b deposition at higher concentrations of serum; a final mAb 3E7 or H17 concentration of 200 μg/ml was sufficient to block activation of the AP by zymosan in 50% NHS (FIG. 2B). This finding should be contrasted with the results seen for the CP (Table 1), in which mAb 3E7 appeared to stabilize C3b/iC3b on B cells.

To determine whether AP activation could induce lysis of a CP substrate, and whether mAb 3E7 might affect its resistance to the AP, the experiment illustrated in FIG. 2C was conducted in the presence of zymosan and 2% sheep EA, a substrate sensitive to the CP. The buffer used contained Mg-EGTA. The sheep EA were not lysed during the 37° C. incubation in 20 or 50% NHS in the presence or absence of mAbs 3E7 or H17. However, in the absence of these inhibitors, C3b was deposited on the zymosan: final concentrations of 100 μg/ml mAb 3E7 or mAb H17 blocked C3b deposition.

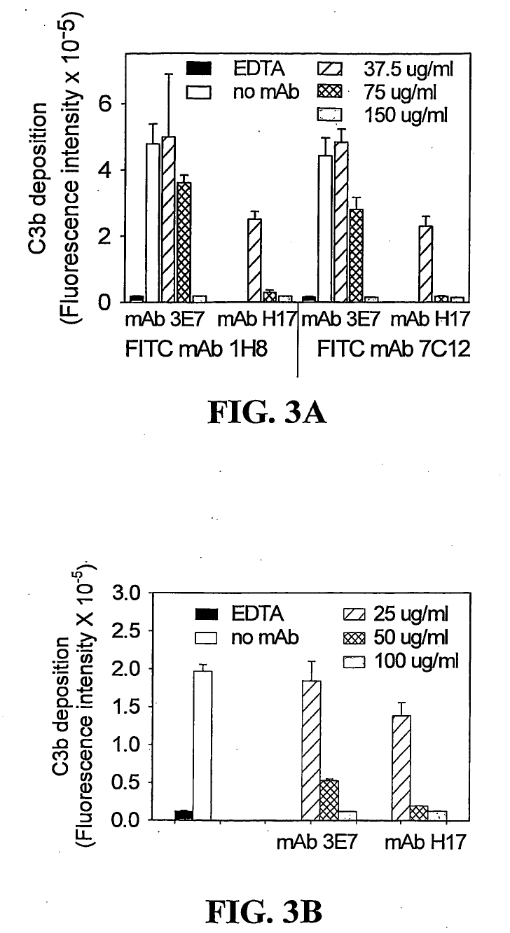

Next examined was the ability of mAbs 3E7 and H17 to block AP activation mediated by Sepharose 4B (FIGS. 3A and 3B). Two different mAbs were used to measure C3b deposition and a pattern of inhibition similar to that seen with zymosan as substrate was found, i.e., virtually complete inhibition can be achieved with final concentrations of 100 μg/ml mAb in 50% NHS (FIG. 3B); in 75% serum, 150 μg/ml mAb completely blocks the AP.

With respect to the mechanism of AP inhibition, an important question is which step or steps in the C3b deposition process and in the generation of the C3b convertase are blocked by mAbs 3E7 and H17? In particular, it is possible that mAb 3E7 may bind to C3(H2O), the “tickover” product that initiates the AP (equation 1) (Muller-Eberhard, H. J., Ann. Rev. Bioch., 1988, 57:321-347; Pangburn, M. K., et al., J. Exp. Med., 1981, 154:856-867). In order to test this hypothesis, we examined the binding of A1488 mAbs 3E7 and H17 to Sepharose 4B under two separate conditions. In the first case, C3b deposition was achieved by incubating NHS with Sepharose 4B for 30 minutes at 37° C.; the opsonized Sepharose 4B was then washed, and the A1488 mAbs were added to assay for C3b-Sepharose.

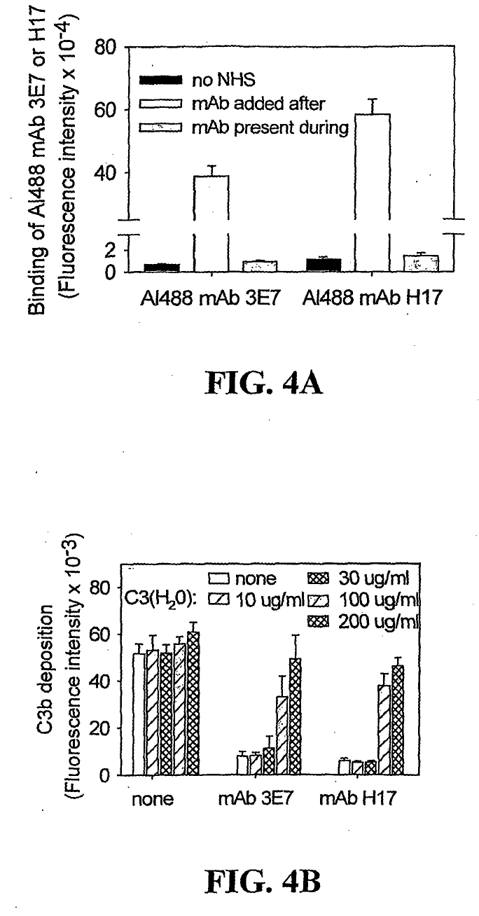

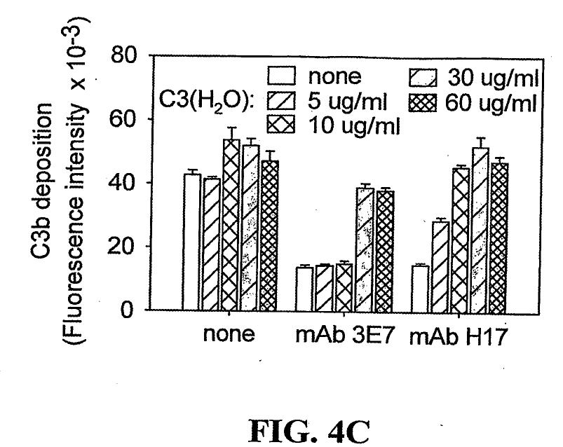

Alternatively, the A1488 mAbs were present in the incubation mixture at the start of the 30 minutes opsonization with NHS (FIG. 4A). In this way it could be determined if moderate amounts of the mAbs are bound to the Sepharose during the opsonization step due to the deposition of some C3b, but additional deposition of C3b is blocked. In fact, we find that in 50% NHS there is little binding of A1488 mAbs 3E7 or H17 to Sepharose 4B if the mAbs are present during the opsonization. However, if the mAbs were added to the washed Sepharose after opsonization, substantial binding of these mAbs was evident; this implies that mAbs 3E7 and H17 bind to C3(H₂O), and then block its binding to factor B, thus preventing generation of any “downstream” C3b. AP-mediated C3b deposition is blocked on Sepharose 4B if the unlabeled mAbs 3E7 or H17 are present during the opsonization step (FIGS. 3A and 3B).

The above-described experiment was also carried out using zymosan in place of Sepharose 4B. Zymosan was added to 50% NHS in Mg-EGTA buffer with and without A1488 mAb 3E7 or H17 (100 μg/ml).

After 30 minutes, the reactions were quenched with EDTA, washed and samples without the mAbs were probed with the respective A1488 mAbs. As with the Sepharose 4B experiment (FIG. 4A), when A1488 mAbs 3E7 and H17 were included at the beginning of the reaction a low level of binding was observed (3300 and 2000 MESF, respectively), but a greater than 200-fold increase in binding was evident when the probes were added to the serum-opsonized zymosan after reaction with NHS (690,000 and 810,000 MESF, respectively). Background values for binding to naïve Sepharose of A1488 mAb 3E7 and A1488 mAb H17 were 500 and 400 MESF units, respectively.

As a further test of the specificity of mAbs 3E7 and H17, C3(H2O) was prepared by incubating NHS or purified C3 with KBr (Oran, A., et al., J. Biol. Chem., 1999, 274:5120-5130; Pangburn, M. K., et al., J. Exp. Med., 1981, 154:856-867), and it was determined whether the treated serum or treated C3 could block the action of the mAbs. Dose-response experiments indicated that pre-incubation of mAbs 3E7 or H17 with both reagents substantially inhibited the ability of the mAbs to block the AP, as defined by the generation of C3b-Sepharose (FIGS. 4B and 4C). In the absence of mAb 3E7 or H17, neither the treated serum or the purified C3(H₂O) alone showed any inhibitory activity, and in fact they may have modestly enhanced the AP. However, both reagents substantially blocked the inhibitory action of mAbs 3E7 and H17. Based on the concentration dependence of the reactions, the most reasonable explanation for these findings is that binding of the C3(H₂O) to mAb 3E7 or to H17 prevented the mAbs from interfering with formation of the AP C3b convertase.

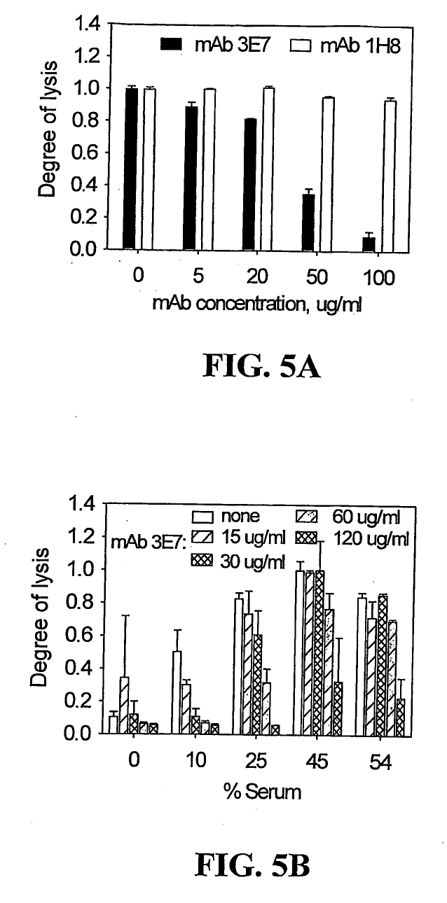

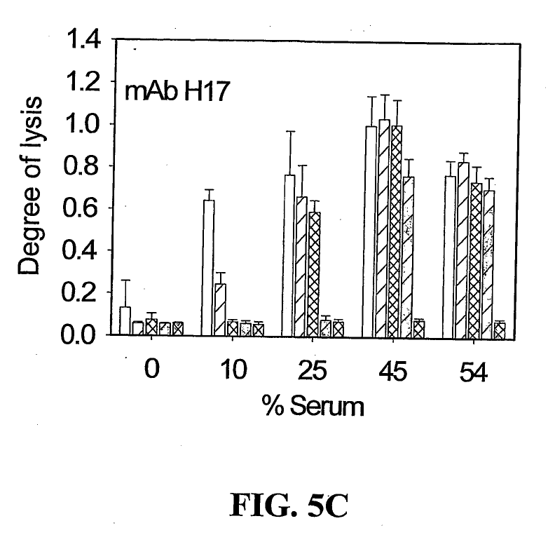

The AP of complement promotes lysis of rabbit E (Pangburn et al., 1983), and in a third independent test we examined the ability of mAbs 3E7 and H17 to prevent lysis of rabbit E in Mg-EGTA NHS. Dose-response experiments indicated that these mAbs protect rabbit E from lysis, and consistent with the previous experiments, it was found that a final concentration of 100 μg/ml mAb is sufficient to block almost all AP lysis of rabbit E in serum (FIGS. 5A, 5B, and 5C). On the other hand, mAb 1H8, which binds to a different C3 fragment epitope than does mAb 3E7/H17, did not protect rabbit E from AP-mediated lysis (FIG. 5A). Several additional mAbs specific for C3b were also developed, and one of them, mAb 2C5, cross-competed with mAb 3E7 for binding to C3b-opsonized substrates, and mAb 2C5 also blocked the AP of complement as effectively as mAb 3E7 (not shown). However, the other anti-C3b mAbs, which did not cross-compete with mAb 3E7/H17, were incapable of blocking the alternative pathway in the three assays described herein. Finally, antibody-opsonized sheep erythrocytes (“EA”), pretreated with specific antibodies, have traditionally been used to test the CP of complement activation (Whaley, K. et al., Eds.: Dodds, A. W. and Sim, R. B., Complement. A Practical Approach, 1997, IRL at Oxford University Press, 19-48). Under conditions allowing CP activation, antibody-opsonized sheep erythrocytes (“EA”) are effectively lysed by NHS, even in the presence of mAb 3E7. For example, in 20% NHS, >95% lysis of antibody-opsonized sheep erythrocytes (“EA”) was obtained in the presence and absence of 100 μg/ml mAb 3E7 or mAb H17.

To assess the generality of the AP blockade, five different individual NHS samples were used in a C3b deposition inhibition assay. Sera were combined with Sepharose 4B in Mg-EGTA buffer in the presence and absence of mAbs 3E7 or H17, following the general approach illustrated in FIG. 3. In all cases, mAb 3E7 or H17, each at 100 μg/ml, quantitatively inhibited deposition of C3b onto Sepharose in 50% NHS, as judged by steady-state fluorescence (not shown).

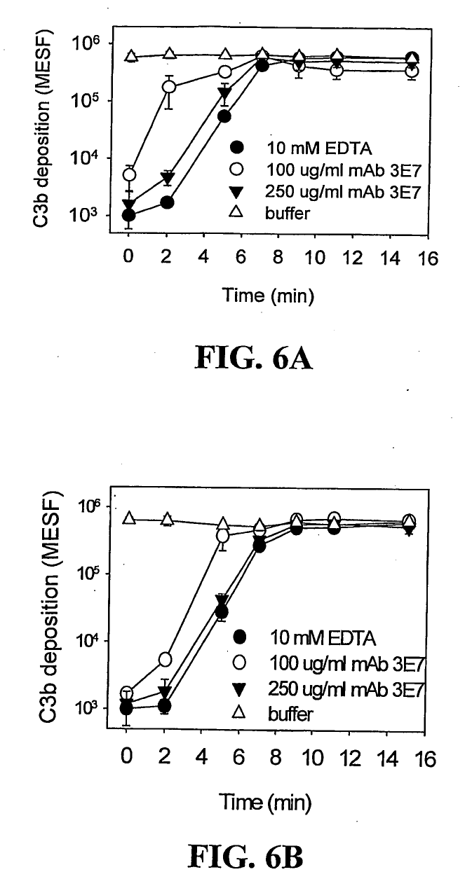

A kinetic assay was developed and described herein to evaluate the efficacy of mAb 3E7 in stopping the alternative pathway once it has started. Aliquots of a reaction mixture containing zymosan and 50% NHS-Mg-EGTA were removed at timed intervals and mixed with buffer, or with mAb 3E7 at 100 or 250 μg/ml mAb; a fourth aliquot was quenched into cold EDTA. As illustrated in FIGS. 6A and 6B, addition of mAb 3E7 led to kinetic profiles of inhibition similar to those observed with cold EDTA, indicating that at sufficiently high concentrations, mAb 3E7 effectively stopped progression of the alternative pathway after it was initiated.

### Antibody Sequences

The antibody sequences of mAb 3E7 and its deimmunized derivative, H17, are provided in FIGS. 7, 8, 9A and 9B. FIG. 7 demonstrates the DNA and amino acid sequence of the 3E7 murine monoclonal antibody heavy chain variable region. Restriction sites and coding regions are indicated.

The amino acid sequence for the 3E7 heavy chain variable region comprises the sequence having SEQ ID NO:1:

(SEQ ID NO:1)

EVQLQESGPSLVKPSQTLSLTCSVTGDSITSDYWNWIRKFPGNKLESMGY
ITYSGTTYYNPSLKSRISITRDTSKNQYYLQLNSVTSEDTATYYCARGVD
YEPSYYFDYWGQGTTLTVSS.

FIG. 8 shows the DNA and amino acid sequence of the 3E7 murine monoclonal antibody light chain variable region, as well as restriction sites and coding regions. The amino acid sequence for the 3E7 light chain variable region comprises a sequence having SEQ ID NO:2:

(SEQ ID NO:2)

DIQMTQTTSSLSASLGDRVTISCSASQGISNYLNWYQQKPDGTVKLLIYY
TSSLHSGVPSRFSGSGSGTDYSLTISNLEPEDIATYYCQQYSNLPWTFGG
GTKLEIK

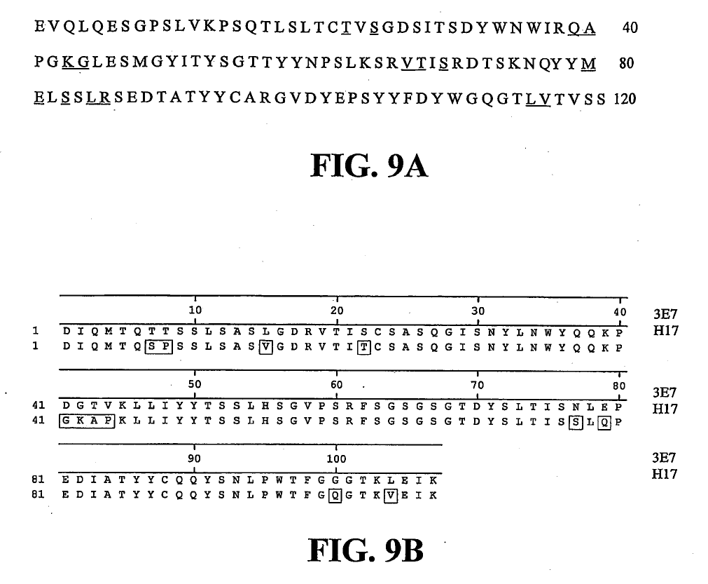

FIGS. 9A and 9B illustrate the heavy chain variable region (9A) and light chain variable region (9B) amino acid sequences of H17. In FIG. 9A, the underlined regions indicate the amino acid residues which have been changed relative to 3E7. FIG. 9B represents the H17 light chain amino acid sequence and also compares the light chain region of H17 to 3E7, with the outlined letters in H117 indicating changes in amino acid residue relative to 3E7.

The H17 heavy chain variable region amino acid sequence comprises a sequence having the sequence of SEQ ID NO:3:

(SEQ ID NO:3)

EVQLQESGPSLVKPSQTLSLTCTVSGDSITSDYWNWIRQAPGKGLESMGY
ITYSGTTYYNPSLKSRVTISRDTSKNQYYMELSSLRSEDTATYYCARGVD
YEPSYYFDYWGQGTLVTVSS.

The H17 light chain variable region amino acid sequence comprises a sequence having the sequence of SEQ ID NO:4:

(SEQ ID NO:4)

DIQMTQSPSSLSASVGDRVTITCSASQGISNYLNWYQQKPGKAPKLLIYY
TSSLHSGVPSRFSGSGSGTDYSLTISSLQPEDIATYYCQQYSNLPWTFGQ
GTKVEIK.

### SUMMARY

The murine monoclonal antibody mAb 3E7 was first identified based on its ability to enhance classical pathway activation and deposition of C3b/iC3b on RTX-opsonized cells Kennedy, A. D., et al., Blood, 2003, 101:1071-1073). It is reasonable to assume that its mechanism of enhancement is based at least in part on its ability to inhibit binding of factor H to C3b-opsonized substrates (FIGS. 1A and 1B); factor H binding normally downregulates complement activation, ultimately resulting in generation of predominantly C3dg fragments (Table 1) (Muller-Eberhard, H. J., Ann. Rev. Bioch., 1988, 57:321-3478; Walport, M. J., N. Engl. J. Med., 2001, 344:1058-1066). In other words, mAbs 3E7 and H17 appear to bind to a site on C3b which would otherwise be occupied by factor H (or factor B) (Becherer, J. D., et al., Biochemistry, 1992, 31:1787-1794; Farries, T. C., et al., Inflamm, 1990, 7:30-41; Koistinen, V., et al., Complement Inflamm., 1989, 6:270-280; Lambris, J. D., et al., J. Immunol., 1996, 156:4821-4832). In fact, other anti-C3b mAbs are reported to block binding of factor H (and factor B) to C3b-opsonized substrates (Becherer, J. D., et al., Biochemistry, 1992, 31:1787-1794; Koistinen, V., et al., Complement Inflamm., 1989, 6:270-280), but there have been no previous attempts to use such mAbs to block the AP.

The present application demonstrates that mAbs 3E7 and H17, specific for C3b/iC3b, can inhibit activation of the AP, based on analysis of AP activation for three different substrates: zymosan, Sepharose 4B, and rabbit E (FIGS. 2, 3, and 5). The most likely mechanism for this effect is that by binding to either C3(H2O) or to C3 fragments, the mAbs can inhibit binding of factor B to these substrates (equations 1 and 2); we have verified that these mAbs block binding of factor B to C3b-opsonized zymosan and Sepharose 4B (FIGS. 1C and 1D).

With respect to the AP, when the mAbs are added to serum during the presumptive opsonization step they show negligible binding to AP substrates (FIG. 4A). Without wishing to be bound by any particular theory, it is believed that the reason for the lack of binding is that the initiating factor in the AP, the “tickover” product C3(H2O) (Muller-Eberhard, H. J., Ann. Rev. Bioch., 1988, 57:321-347; Pangburn, M. K., et al., J. Exp. Med., 1981, 154:856-867), is immediately bound by the mAbs, thus preventing binding of factor B to C3(H2O), and therefore largely inhibiting any downstream C3b deposition. It is important to recognize that after AP-mediated deposition of C3b on the substrates, mAbs 3E7 and H17 manifest strong binding as a result of increased C3b deposition. Further evidence supporting the interaction between C3(H₂O) and these mAbs is the observation that addition of a source of C3(H₂O) to mAbs 3E7 or H17 blocks their ability to inhibit the AP (FIGS. 4B and 4C).

The results clearly demonstrate that mAb 3E7 can block ongoing complement activation (FIG. 6), but that more of the mAb is needed (250 μg/ml), presumably because substantial amounts of the mAb are bound to “decoys” of soluble or substrate bound C3b, thus limiting the effectiveness of the mAb at lower concentrations.

The in vitro findings disclosed herein provide substantial evidence that a mAb specific for C3b (or C3(H₂O)) can selectively block the AP, most likely by binding to C3(H₂O) and therefore inhibiting an activation step in the pathway, binding of factor B to C3(H₂O).

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. The appended claims are intended to be construed to include all such embodiments and equivalent variations. Accordingly, the present invention is not intended to be limited to the embodiments shown herein but is to be accorded the widest scope consistent with the principles and novel features disclosed herein.

What is claimed is:

1. A method of inhibiting the alternative complement pathway in a subject, said method comprising administering to said subject a pharmaceutical composition comprising an effective amount of at least one monoclonal antibody, or a derivative, fragment, or homolog thereof, , and a pharmaceutically-acceptable carrier, thereby inhibiting the alternative complement pathway in a subject.

2. The method of claim 1, wherein said antibody is directed against at least one peptide selected from the group consisting of C3, C3(H₂O), and C3b.

3. The method of claim 1, wherein said monoclonal antibody, and fragments, derivatives, and homologs thereof, is selected from the group consisting of a murine monoclonal antibody, a humanized murine monoclonal antibody, a deimmunized murine monoclonal, and a human monoclonal antibody.

4. The method of claim 3, wherein said antibody is 3E7 or H17.

5. The method of claim 1, wherein said antibody comprises amino acid sequences selected from the group consisting of SEQ ID NOs:1, 2, 3, and 4.

6. The method of claim 1, wherein said method inhibits the alternative complement pathway.

7. The method of claim 6, wherein said method inhibits the activation or progression of the alternative complement pathway.

8. The method claim 6, wherein said method inhibits C3b/iC3b degradation to C3dg.

9. The method of claim 7, wherein said method inhibits factor H binding to a peptide selected from the group consisting of C3, C3(H₂O), and C3b.

10. The method of claim 7, wherein said method inhibits factor B binding to a peptide selected from the group consisting of C3, C3(H₂O), and C3b.

11. A method of treating an alternative complement pathway mediated disease, disorder, or condition in a subject in need thereof, said method comprising administering to said subject a pharmaceutical composition comprising an effective amount of at least one inhibitor of said alternative complement pathway and a pharmaceutically-acceptable carrier, thereby treating an alternative complement pathway mediated disease, disorder, or condition.

12. The method of claim 11, wherein said inhibitor is an antibody, or derivatives, fragments, and homologs thereof.

13. The method of claim 12, wherein said antibody is a monoclonal antibody.

14. The method of claim 13, wherein said monoclonal antibody, and derivatives, fragments, and homologs thereof, is selected from the group consisting of a murine monoclonal antibody, a humanized murine monoclonal antibody, a deimmunized murine monoclonal antibody, and a human monoclonal antibody.

15. The method of claim 14, wherein said antibody is 3E7 or H17.

16. The method of claim 13, wherein said antibody comprises at least one amino acid sequence selected from the group consisting of SEQ ID NOs:1, 2, 3, and 4.

17. The method of claim 11, wherein said method inhibits binding of factor B or factor H to a peptide selected from the group of peptides consisting of C3, C3(H₂O), and C3b.

18. The method of claim 11, wherein said subject is selected from the group of animals consisting of cattle, pigs, horses, sheep, cats, dogs, birds, non-human primates, and humans.

19. The method of claim 11, wherein said disease, disorder, or condition is associated with inflammation or ischemia.

20. The method of claim 19, wherein said disease, disorder, or condition associated with ischemia or inflammation is selected from the group consisting of inflammatory diseases, ischemia reperfusion injury, kidney injury, cardiac injury, myocardial infarction, transplantation, and cardiopulmonary bypass.

21. An isolated nucleic acid comprising a nucleic acid sequence encoding at least one chain of an antibody which binds to a peptide selected from the group consisting of C3, C3(H₂O), and C3b, wherein said antibody inhibits the alternative complement pathway.

22. The isolated nucleic acid of claim 21, wherein said nucleic acid comprises a nucleic acid sequence encoding an amino acid sequence selected from the group consisting of SEQ ID NOs:1, 2, 3, and 4, and derivatives, fragments, and homologs thereof.

23. A vector comprising the isolated nucleic acid of claim 21.

24. A recombinant cell comprising the vector of claim 21.

25. An antibody, and derivatives, fragments, and homologs thereof, which binds to at least one of the peptides selected from the group of peptides consisting of C3, C3(H₂O), and C3b, wherein said binding of said antibody inhibits the alternative complement pathway.

26. The antibody of claim 25, wherein said antibody is a monoclonal antibody.

27. A hybridoma comprising a nucleic acid sequence encoding the monoclonal antibody of claim 26.

28. The monoclonal antibody of claim 27, wherein said monoclonal antibody is 3E7, or a derivative, fragment, or homolog thereof.

29. A pharmaceutical composition comprising an antibody of claim 25 and a pharmaceutically-acceptable carrier.

30. A kit for inhibiting the alternative complement pathway, said kit comprising a pharmaceutical composition comprising at least one antibody directed against a peptide selected from the group consisting of C3, C3(H₂O), and C3b, and a pharmaceutically-acceptable carrier, an applicator, and an instructional material for the use thereof.
